(19) United States
(12) Patent Application Publication
(10) Pub. No.: US 2026/0198796 A1
(43) Pub. Date: Jul. 16, 2026

(54) METHOD FOR EVALUATING SEGMENTAL BODY COMPOSITION DATA OF HUMAN BODY USING BIOELECTRICAL IMPEDANCE VECTOR ANALYSIS

(71) Applicant: STARBIA MEDITEK CO., LTD., TAICHUNG CITY (TW)

(72) Inventor: KUEN-CHANG HSIEH, TAICHUNG CITY (TW)

(21) Appl. No.: 19/141,901

(22) PCT Filed: Mar. 4, 2024
(86) PCT No.: PCT/CN2024/079911

§ 371 Date: Jun. 20, 2025

(30) Foreign Application Priority Data
Feb. 5, 2024 (CN) 202410161228.2

(51) Int. Cl. A61B 5/0537

(52) U.S. Cl. CPC A61B 5/0537

(57) ABSTRACT

A method for evaluating segmental body composition data of a human body using bioelectrical impedance vector analysis includes steps of providing a plurality of R-Xc graphs for body segments of a population group, wherein each of the R-Xc graphs has an X-axis representing a standardized resistance, and a Y-axis representing a standardized reactance, using a body composition analyzer to measure a standardized resistance and a standardized reactance for each body segment of a subject, and mapping the standardized resistance and the standardized reactance of the subject to a coordinate in the corresponding R-Xc graph. As such, an index indicative of the subject's body composition in each body segment relative to the physiological characteristics of the population group can be evaluated.

### FIELD OF THE INVENTION

The present invention relates to bioelectrical impedance vector analysis technology and more particularly, to a method for evaluating segmental body composition data of a human body by using bioelectrical impedance vector analysis.

### DESCRIPTION OF THE RELATED ART

Bioelectrical Impedance Vector Analysis (BIVA) is a technique used to evaluate human body composition. It involves passing a weak electrical current through the human body to understand the distribution between intracellular fluid (ICF) and extracellular fluid (ECF). Human body composition mainly includes muscle, fat, and bone. Currently, a common method for evaluating body composition data is illustrated in FIG. 1, where bar charts are used for assessment. For example, in the case of evaluating skeletal muscle mass, a first bar chart is generated by statistically quantifying muscle mass. This first bar chart serves as a comparison benchmark and is classified into categories such as low, normal, or high muscle mass. After a subject's skeletal muscle mass is measured using BIVA, the result is quantified into a second bar chart. By comparing this second bar chart with the first bar chart, it is possible to assess whether the subject's muscle mass is low, normal, or high.

However, the above evaluating method cannot evaluate how the subject's skeletal muscle mass compares to the physiological characteristics of a specific population group (such as athletes, elderly individuals, people in the same geographical location, those with the same physiological traits, or a specific age group). Therefore, the conventional methods for evaluating body composition data of various body segments of the human body still have room for improvement.

### SUMMARY OF THE INVENTION

It is one objective of the present invention to provide a method for evaluating segmental body composition data of a human body by using bioelectrical impedance vector analysis, which can evaluate an index indicative of a subject's body composition in each body segment relative to physiological characteristics of a population group, thereby enhancing the industrial applicability.

To attain the above objective, the method of the present invention comprises following steps. A computing unit is used to provide a plurality of R-Xc graphs for body segments of a population group, wherein the body segments represented by the R-Xc graphs comprise at least two of the following: whole body, a right upper limb, a left upper limb, a right lower limb, a left lower limb, and a trunk. Each of the R-Xc graphs comprises an X-axis, a Y-axis, a first vector line, a second vector line, and a plurality of tolerance intervals. The X-axis represents a standardized resistance (Z(R/G1)) of the population group after Z-transformation. The Y-axis represents a standardized reactance (Z(Xc/G1)) of the population group after Z-transformation. The X-axis and the Y-axis intersect to form an origin, and the X-axis and Y-axis define a first quadrant, a second quadrant, a third quadrant, and a fourth quadrant, each of which represents a body composition state. The first vector line and the second vector line intersect each other and pass through the origin. The plural tolerance intervals are annular in shape and concentrically arranged from the origin outward, and the plural tolerance intervals are expressed in percentage units, with the values increasing progressively in a direction away from the origin, and the plural tolerance intervals cover the first, second, third, and fourth quadrants. After that, a body composition analyzer is used to measure a standardized resistance (Z(R/G2)) and a standardized reactance (Z(Xc/G2)) for each body segment of a subject. By mapping the standardized resistance (Z(R/G2)) and the standardized reactance (Z(Xc/G2)) of the subject to a coordinate in the corresponding R-Xc graph, the subject's body composition data is evaluated based on the quadrant in which the coordinate is located and the tolerance interval into which the coordinate falls

It can be seen from the above that the method of the present invention can evaluate an index indicative of the subject's body composition in each body segment relative to physiological characteristics of the population group, thereby enhancing the industrial applicability.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a method for evaluating body composition data in the prior art.

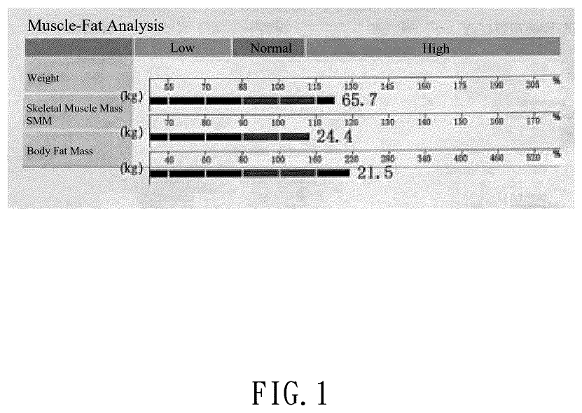

FIG. 2 is a block diagram of a method of the present invention.

FIG. 3 is a first R-Xc graph provided by a first embodiment of the method of the present invention.

FIG. 4 is a second R-Xc graph provided by the first embodiment of the method of the present invention.

FIG. 5 is a third R-Xc graph provided by the first embodiment of the method of the present invention.

FIG. 6 is a fourth R-Xc graph provided by the first embodiment of the method of the present invention.

FIG. 7 is a fifth R-Xc graph provided by the first embodiment of the method of the present invention.

FIG. 8 is a sixth R-Xc graph provided by the first embodiment of the method of the present invention.

FIG. 9 is a first R-Xc graph provided by a second embodiment of the method of the present invention.

FIG. 10 is a second R-Xc graph provided by the second embodiment of the method of the present invention.

FIG. 11 is a third R-Xc graph provided by the second embodiment of the method of the present invention.

FIG. 12 is a fourth R-Xc graph provided by the second embodiment of the method of the present invention.

FIG. 13 is a fifth R-Xc graph provided by the second embodiment of the method of the present invention.

FIG. 14 is a sixth R-Xc graph provided by the second embodiment of the method of the present invention.

### DESCRIPTION OF THE MARKINGS WITH DRAWINGS

10: Bioimpedance vector analysis technique to evaluate the body composition data of various human limbs
20: Human limb R-Xc diagram
21: First R-XC Figure
22: Second R-Xc Figure
23: Third R-Xc Figure
24: Fourth R-Xc Figure
25: Fifth R-Xc Figure
26: Sixth R-Xc Figure
30: X Coordinate axis
40: Y axis
41: Quadrant 1
42: Second quadrant
43: Third quadrant
44: Quadrant 4
50: First vector line
60: Second vector line
70: Allowable interval
71: 50% Allowable Interval
72: 75% Allowable Interval
73: 90% Allowable Interval
80: Origin
90: Contour line
91: First Contour
92: Second Contour
93: Third Contour
100: Marking
P: Coordinate point
21′: 1st R-Xc Figure
22′: 2nd R-Xc Figure
23′: 3rd R-Xc Figure
24′: Fourth R-Xc Figure
25′: Fifth R-Xc Figure
26′: Sixth R-Xc Figure
70′: Allowable interval
71′: 50% allowable interval
72′: 75% allowable interval
73′: 90% allowable interval
80′: origin
90′: Contour
91′: First Contour
92′: 2nd Contour
93′: 3rd Contour.

### DETAILED DESCRIPTION OF THE INVENTION

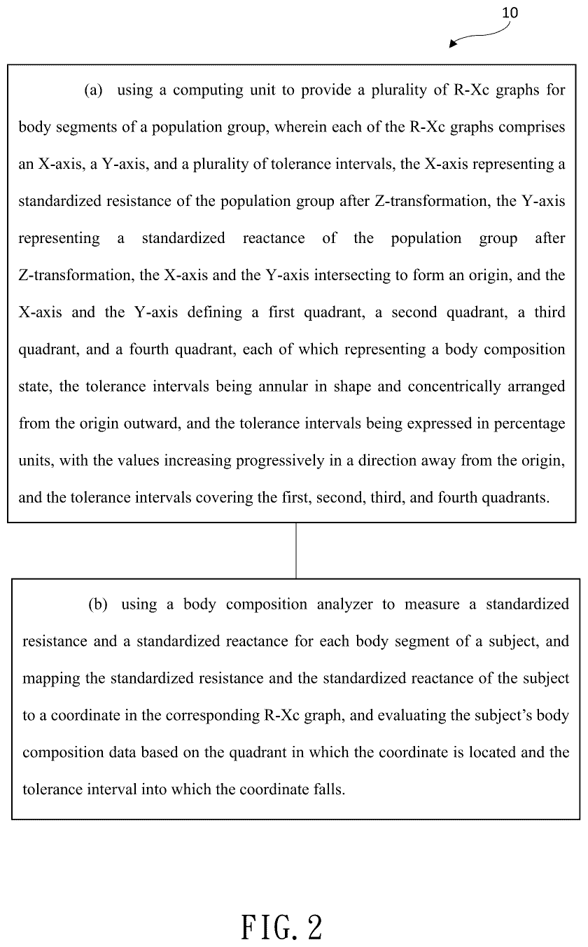

FIG. 2 shows a method 10 for evaluating segmental body composition data of a human body by using bioelectrical impedance vector analysis. The body composition data is exemplified using soft tissue, which includes skin, subcutaneous fat, muscle, tendons, tendon sheaths, ligaments, fascia, synovial membranes, bursae, joint capsules, peripheral nerves, fibrous tissues, lymphatic tissues, vascular tissues, and other connective tissues, but excludes bones and fat. However, in practical use, if necessary for evaluation purposes, bones or fat may also be used as the basis for the body composition data.

As shown in FIG. 2, the method 10 of the present invention comprises following steps.

(a) using a computing unit to provide a plurality of R-Xc graphs 20 for body segments of a population group, wherein each of the R-Xc graphs 20 comprises an X-axis 30, a Y-axis 40, a first vector line 50, a second vector line 60, and a plurality of tolerance intervals 70. The X-axis 30 represents a standardized resistance (Z(R/G1)), wherein R denotes resistance. The Y-axis 40 represents a standardized reactance (Z(Xc/G1)), wherein Xc denotes reactance. The tolerance interval 70 is defined based on the sample mean x̅ as an estimate of the population group mean μ, and the sample standard deviation S as an estimate of the population group standard deviation σ. This interval is used to estimate individual values falling within the specified range, which is referred to as the tolerance interval.

In the first embodiment, the population group is exemplified by middle-aged and elderly individuals (aged 45 to 65). The plurality of the R-Xc graphs 20 are generated by measuring a large number of the middle-aged and elderly individuals (more than one hundred, including various occupations, genders, geographic regions, etc.) with respect to the whole body, right upper limb, left upper limb, right lower limb, left lower limb, and trunk. The resistance and reactance obtained through measurement are standardized by the height of the population group to yield a plurality of parameters (R/G1 and Xc/G1), wherein the height serves as a first geometric parameter G1. Subsequently, each of the parameters (R/G1 and Xc/G1) is further normalized to be dimensionless, thereby obtaining the plurality of the R-Xc graphs 20. In practice use, the population group may also be selected, as needed, from athletes, elderly individuals, persons from the same geographic location, persons with the same physiological characteristics, or persons within a specific age group, and thus the implementation of the population group is not limited to the embodiment described herein.

The X-axis 30 and the Y-axis 40 intersect to form an origin 80, wherein the origin 80 represents an intersection point of the average values of R/G1 and Xc/G1. The X-axis 30 and Y-axis 40 define a first quadrant 41, a second quadrant 42, a third quadrant 43, and a fourth quadrant 44, each of which represents a body composition state. The first vector line 50 and the second vector line 60 intersect each other and pass through the origin 80. The plural tolerance intervals 70 are annular in shape and concentrically arranged from the origin 80 outward, and the plural tolerance intervals 70 are expressed in percentage units, with the values increasing progressively in a direction away from the origin 80. The plural tolerance intervals 70 cover the first, second, third, and fourth quadrants 41, 42, 43, 44.

In the first embodiment, the computing unit is exemplified as a body composition analyzer, and the plurality of the R-Xc graphs 20 are displayed on a display screen of the body composition analyzer. In actual use, the computing unit may alternatively be a computer, a tablet, or other hardware device having computing functionality. Therefore, the implementation form of the computing unit is not limited to the embodiment described herein In the first embodiment, the plurality of the R-Xc graphs 20 respectively comprise a first R-Xc graph 21, a second R-Xc graph 22, a third R-Xc graph 23, a fourth R-Xc graph 24, a fifth R-Xc graph 25, and a sixth R-Xc graph 26. The first to sixth R-Xc graphs 21 to 26 respectively represent body segments corresponding to the whole body, the right upper limb, the left upper limb, the right lower limb, the left lower limb, and the trunk. In actual use, the number of the R-Xc graphs 20 may be varied as needed, or the order or arrangement of the human body segments represented by the R-Xc graphs 20 may be modified. Accordingly, both the number of the R-Xc graphs 20 and the human body segments represented by the first to sixth R-Xc graphs 21 to 26 are not limited to the embodiment described herein.

In the first embodiment, the body composition statuses represented by the first quadrant 41, the second quadrant 42, the third quadrant 43, and the fourth quadrant 44 respectively correspond to a low moisture ratio, a high soft tissue ratio, a high moisture ratio, and a low soft tissue ratio. In actual implementation, when bones or fat is used as the basis for bioelectrical impedance vector analysis to evaluate the body composition data of each body segment, the significance represented by the first to fourth quadrants 41 to 44 may vary depending on the specific bioelectrical impedance vector analysis applied to assess the body composition data of the respective body segments In the first embodiment, the tolerance intervals 70 are divided from the origin 80 outward into a 50% tolerance interval 71, a 75% tolerance interval 72, and a 90% tolerance interval 73. In actual implementation, the number and percentages of the tolerance intervals 70 may be modified as needed. Therefore, the number and percentages of the tolerance intervals 70 are not limited to the embodiment described herein.

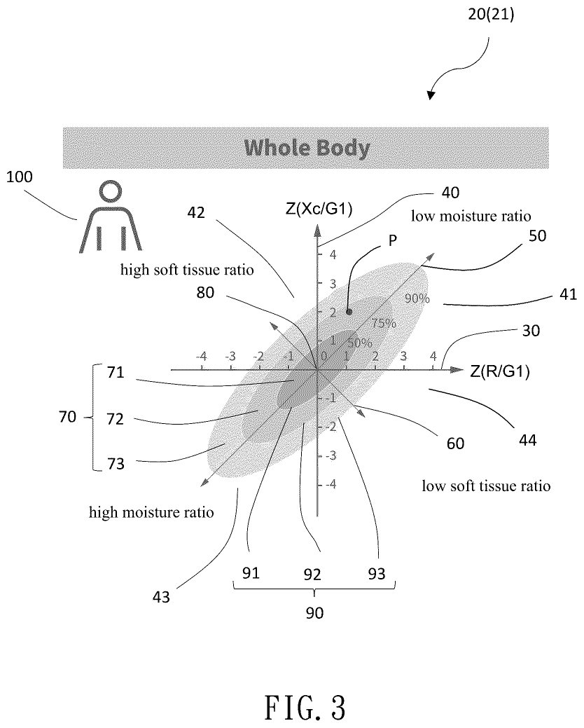

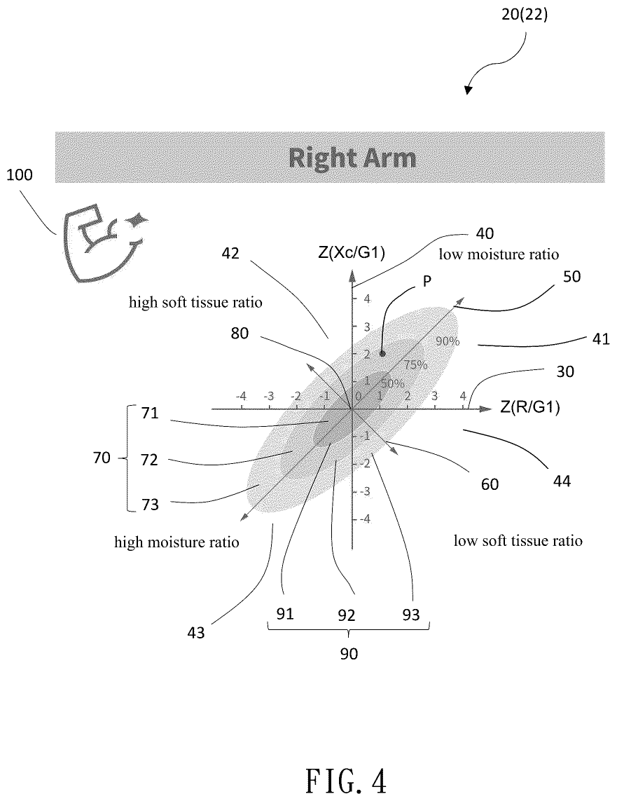

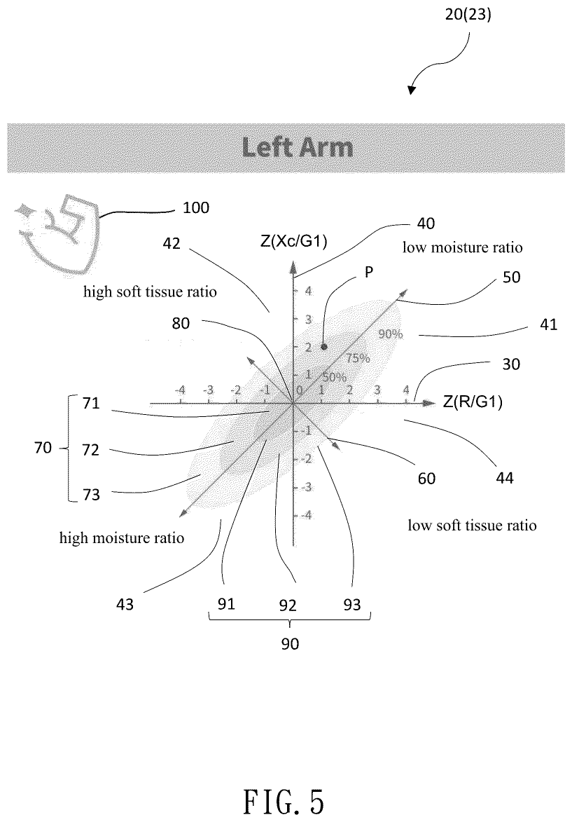

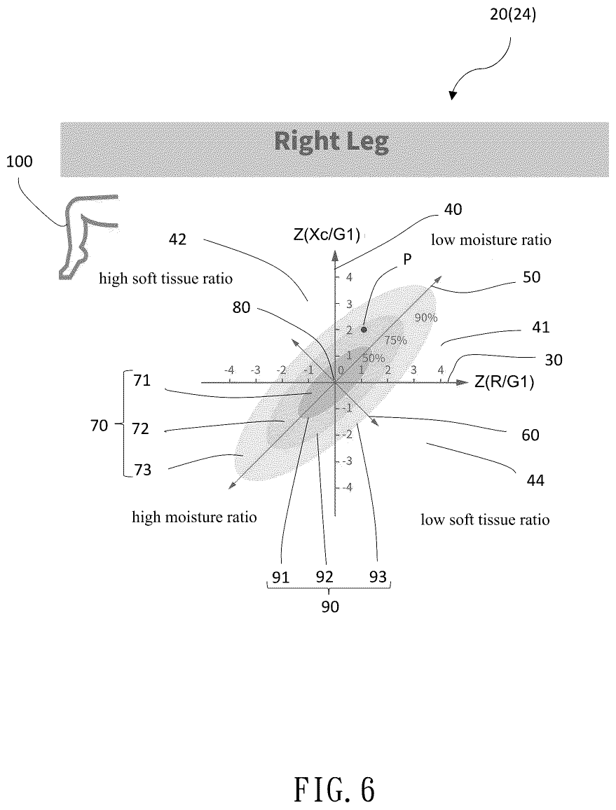

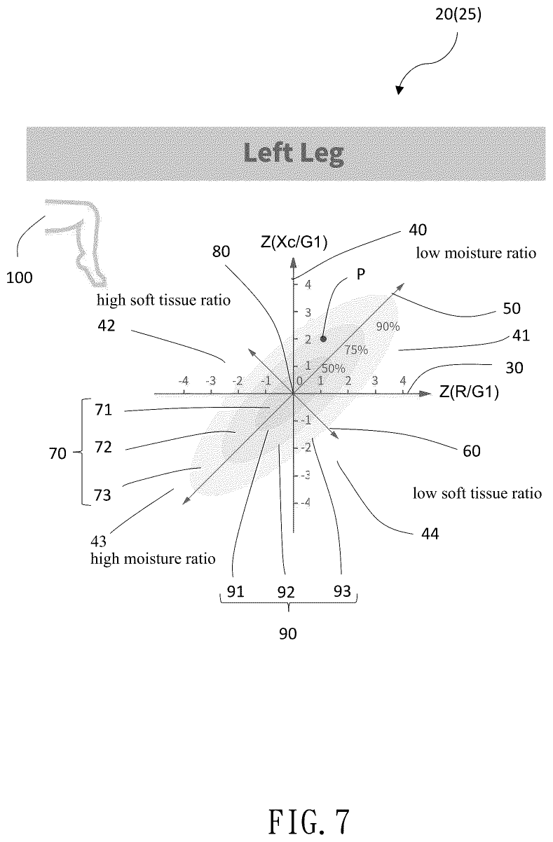

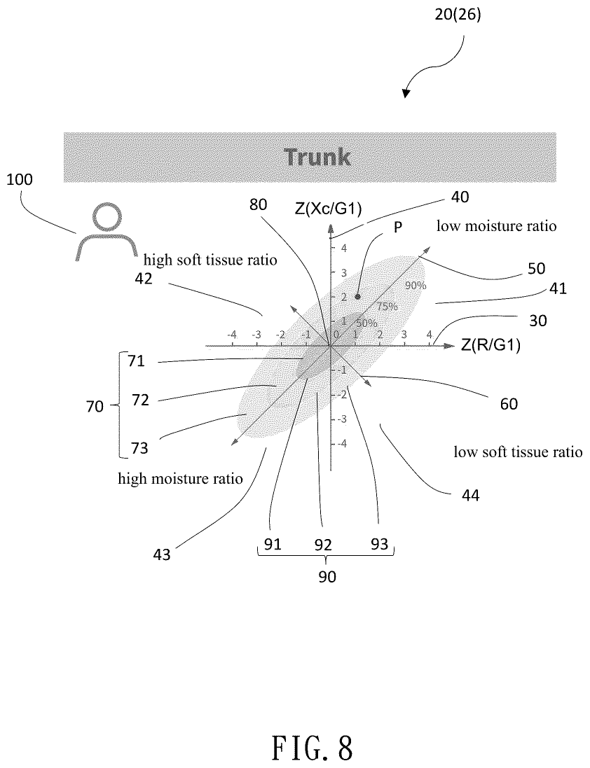

In the first embodiment, as shown in FIGS. 3 to 8, the 50% tolerance interval 71, the 75% tolerance interval 72, and the 90% tolerance interval 73 are respectively delineated by contour lines 90. The contour line 90 closest to the origin 80 is defined as a first contour line 91, the contour line 90 farthest from the origin 80 is defined as a third contour line 93, and the contour line located between the first contour line 91 and the third contour line 93 is defined as a second contour line 92. The area enclosed by the first contour line 91 around the origin 80 corresponds to the 50% tolerance interval 71. The area between the first contour line 91 and the second contour line 92 corresponds to the 75% tolerance interval 72. The area between the second contour line 92 and the third contour line 93 corresponds to the 90% tolerance interval 73. The plurality of the tolerance intervals 70 are each provided with a color, wherein the color transitions from dark to light from the 50% tolerance interval 71 to the 90% tolerance interval 73. Each contour line 90 is formed based on the color difference between adjacent tolerance intervals 71 to 73, thereby facilitating visual distinction among the respective tolerance intervals 71 to 73.

In the first embodiment, each of the R-Xc graphs 20 includes an indication region 100. The indication region 100 respectively represent graphical depictions of the whole body, the right upper limb, the left upper limb, the right lower limb, the left lower limb, and the trunk, and correspond individually to the first to sixth R-Xc graphs 21 to 26. This facilitates easier understanding of the muscle condition of each body segment of the subject represented by the first to sixth R-Xc graphs 21 to 26. However, the indication region 100 can be omitted according to actual needs.

(b) using the body composition analyzer to measure a standardized resistance (Z(R/G2)) and a standardized reactance (Z(Xc/G2)) for each body segment of a subject, and mapping the standardized resistance (Z(R/G2)) and the standardized reactance (Z(Xc/G2)) of the subject to a coordinate P in the corresponding R-Xc graph 20. By determining whether the coordinate P is located within the first quadrant 41, the second quadrant 42, the third quadrant 43, or the fourth quadrant 44, and whether the coordinate point P falls within the 50% tolerance interval 71, the 75% tolerance interval 72, or the 90% tolerance interval 73, it is possible to evaluate which of the tolerance intervals 70 the muscle mass of the subject falls within, relative to that of the population group, thereby enabling the subject to further understand their own muscle condition.

In the first embodiment, the second geometric parameter G2 is exemplified by the height of the subject, such that the resistance-to-second geometric parameter ratio (R/H2) and the reactance-to-second geometric parameter ratio (Xc/H2) are respectively mapped to the R-Xc graph 20, thereby serving as a basis for evaluating the body composition data of the subject.

Although, according to Ohm's law, both the limb length and circumference of a human body segment contribute to electrical resistance, in the present embodiment, for the sake of ease of measurement, the height of the population group is directly used as the first geometric parameter G1, and the height of the subject is used as the second geometric parameter G2. In actual implementation, without considering the difficulty of obtaining data, the height and the segment circumference of the population group, the segment length and the segment circumference of the population group, or the height, the segment length, and the segment circumference of the population group may be alternatively used as the first geometric parameter G1, and further, the height and the segment circumference of the subject, the segment length and the segment circumference of the subject, or the height, the segment length, and the segment circumference of the subject may be alternatively used as the second geometric parameter G2. It is noted that the measurement methods are not limited to imaging or tomography techniques. Therefore, the implementation forms of the first and second geometric parameters G1, G2 are not limited to this embodiment.

The above description sets forth the technical features of the method provided in the first embodiment. The following describes the conditions observed during actual evaluation.

As shown in FIGS. 3 to 7, the resistance and reactance measured from each body segment of the subject are respectively mapped to the first to sixth R-Xc graphs 21 to 26. It can be observed that each coordinate P lies within the first quadrant 41 and the 75% tolerance interval 72 of the corresponding R-Xc graphs 21 to 26. Based on this, it can be evaluated that the muscle composition of the subject's whole body, right upper limb, left upper limb, right lower limb, left lower limb, and trunk exhibits a relatively low moisture ratio. Furthermore, since each coordinate P is located in the first quadrant 41 of the respective R-Xc graphs 21 to 26, it can be further assessed that the body composition of each body segment of the subject corresponds to the 75% and approaches the 90% population distribution.

Accordingly, the method 10 as provided by the first embodiment of the present invention utilizes the first to sixth R-Xc graphs 21 to 26, which respectively represent the whole body, the right upper limb, the left upper limb, the right lower limb, the left lower limb, and the trunk, the first to fourth quadrants 41 to 44, which respectively represents a low moisture ratio, a high soft tissue ratio, a high moisture ratio, and a low soft tissue ratio, and the 50% tolerance interval 71, the 75% tolerance interval 72, and the 90% tolerance interval 73 to evaluate of the subject's muscle mass in comparison with that of the population group, thereby allowing the subject to gain a clearer understanding of their own muscular condition.

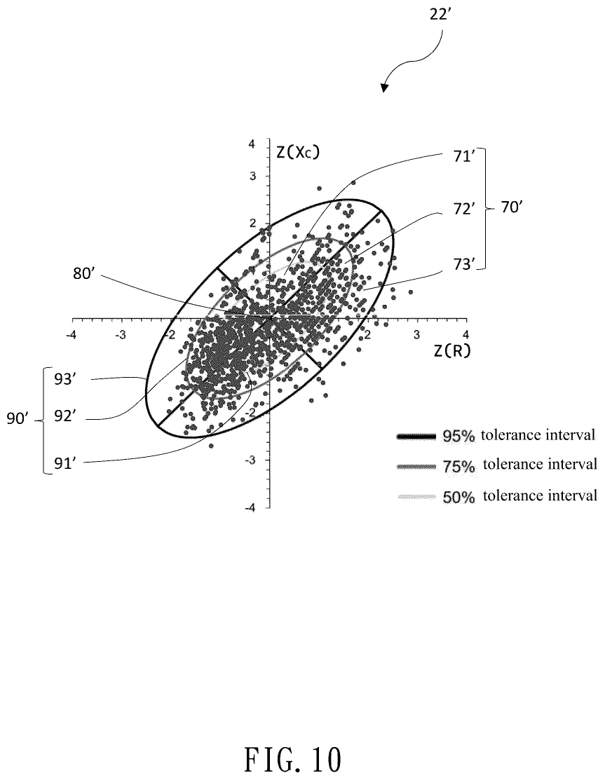

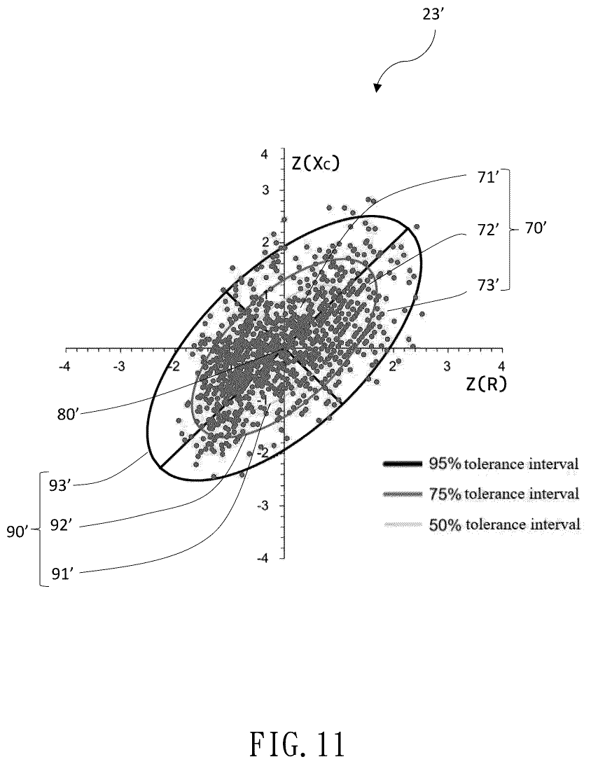

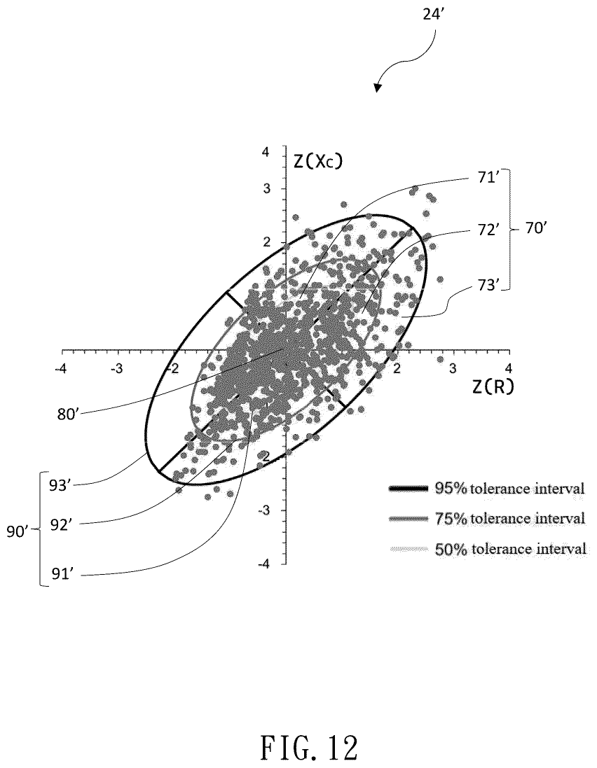

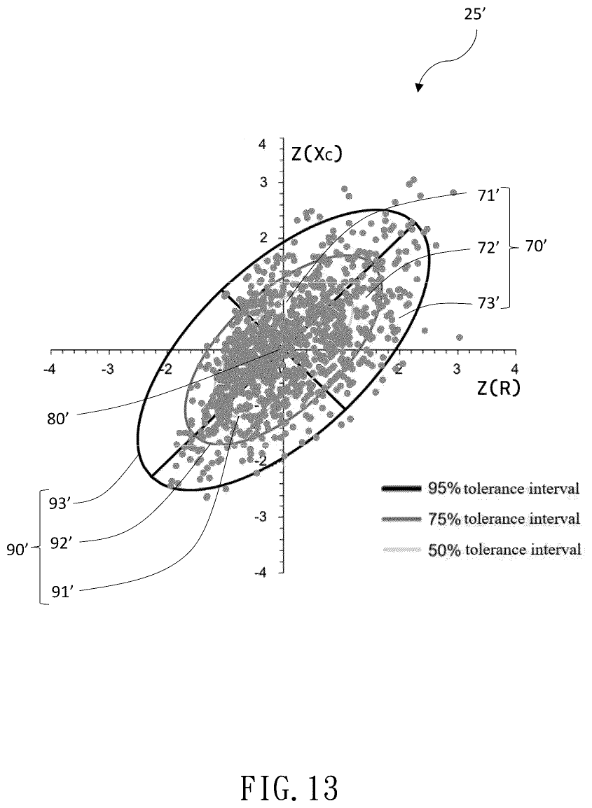

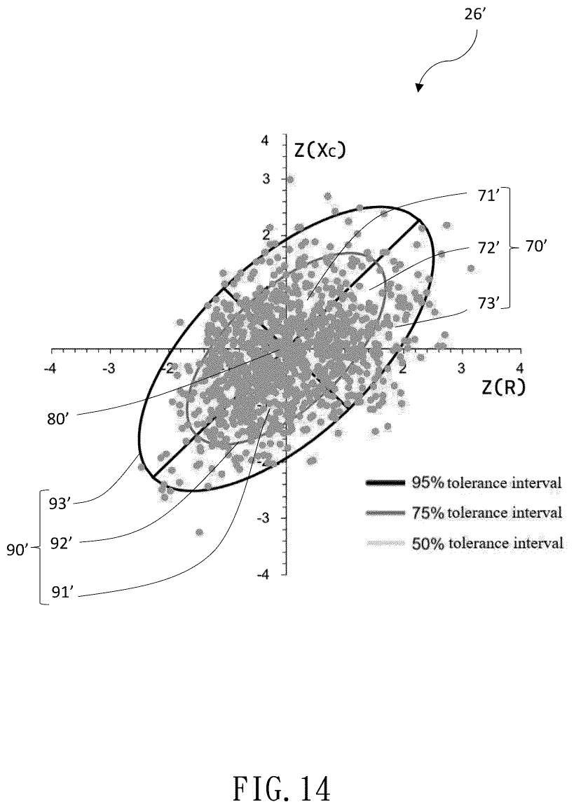

As shown in FIGS. 2 and 9 to 14, the method 10 of the second embodiment is approximately the same with the first embodiment, except for the following differences:

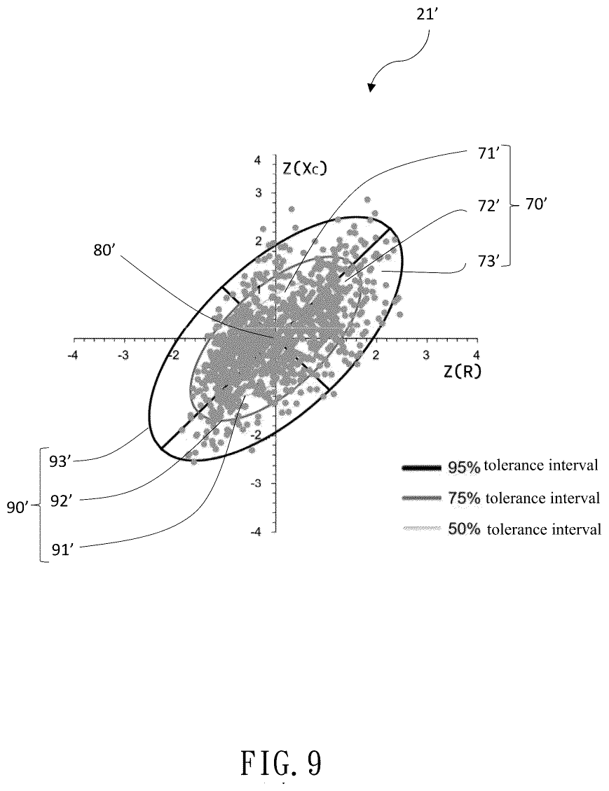

In the second embodiment, as shown in FIGS. 9 to 14, the coordinates measured from the whole body, the right upper limb, the left upper limb, the right lower limb, the left lower limb, and the trunk of a large number of the subjects are all displayed in the first to sixth R-Xc graphs 21′, 22′, 23′, 24′, 25′, and 26′, respectively.

In the second embodiment, the plural tolerance intervals 70′ are divided from the origin 80′ outward into a 50% tolerance interval 71′, a 75% tolerance interval 72′, and a 95% tolerance interval 73′.

In the second embodiment, each of the tolerance intervals 70′ is delineated by a contour line 90′, and the plurality of the contour lines 90′ are each provided with a color. The color becomes progressively darker from the contour line 90′ closest to the origin 80′ toward the outer regions. Among them, the first contour line 91′ is light gray, the second contour line 92′ is gray, and the third contour line 93′ is black.

The remaining technical features and advantageous effects of the second embodiment are the same as those of the aforementioned first embodiment and thus will not be redundantly described herein.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method for evaluating segmental body composition data of a human body using bioelectrical impedance vector analysis, comprising steps of:
(a) using a computing unit to provide a plurality of R-Xc graphs for body segments of a population group, each of the R-Xc graphs comprising an X-axis, a Y-axis, a first vector line, a second vector line, and a plurality of tolerance intervals, the X-axis representing a standardized resistance (Z(R/G1)) of the population group after Z-transformation, the Y-axis representing a standardized reactance (Z(Xc/G1)) of the population group after Z-transformation, the X-axis and the Y-axis intersecting to form an origin, and the X-axis and the Y-axis defining a first quadrant, a second quadrant, a third quadrant, and a fourth quadrant, each of which representing a body composition state, the first vector line and the second vector line intersecting each other and passing through the origin, the plural tolerance intervals being annular in shape and concentrically arranged from the origin outward, and the plural tolerance intervals being expressed in percentage units, with the values increasing progressively in a direction away from the origin, and the plural tolerance intervals covering the first, second, third, and fourth quadrants; and
(b) using a body composition analyzer to measure a standardized resistance (Z(R/G2)) and a standardized reactance (Z(Xc/G2)) for each body segment of a subject, and mapping the standardized resistance (Z(R/G2)) and the standardized reactance (Z(Xc/G2)) of the subject to a coordinate in the corresponding R-Xc graph, and evaluating the subject's body composition data based on the quadrant in which the coordinate is located and the tolerance interval into which the coordinate falls.

2. The method as claimed in claim 1, wherein the plurality of the R-Xc graphs respectively comprise a first R-Xc graph, a second R-Xc graph, a third R-Xc graph, a fourth R-Xc graph, a fifth R-Xc graph, and a sixth R-Xc graph.

3. The method as claimed in claim 2, wherein the first R-Xc graph, the second R-Xc graph, the third R-Xc graph, the fourth R-Xc graph, the fifth R-Xc graph, and the sixth R-Xc graph respectively represent body segments corresponding to the whole body, the right upper limb, the left upper limb, the right lower limb, the left lower limb, and the trunk.

4. The method as claimed in claim 1, wherein the body composition data pertains to soft tissue, and the body composition statuses represented by the first quadrant, the second quadrant, the third quadrant, and the fourth quadrant respectively correspond to a high soft tissue ratio, a low moisture ratio, a low soft tissue ratio, and a high moisture ratio.

5. The method as claimed in claim 1, wherein the plurality of the tolerance intervals are divided from the origin outward into a 50% tolerance interval, a 75% tolerance interval, and a 90% tolerance interval.

6. The method as claimed in claim 1, wherein each of the R-Xc graphs includes an indication region configured to indicate the corresponding body segment.

7. The method as claimed in claim 1, wherein G1 comprises one or a combination of height, segment length, and segment circumference of the population group, and G2 comprises one or a combination of height, segment length, and segment circumference of the subject.

8. The method as claimed in claim 1, wherein the plurality of the tolerance intervals are divided from the origin outward into a 50% tolerance interval, a 75% tolerance interval, and a 95% tolerance interval.
